(19) United States
(12) Patent Application Publication
(10) Pub. No.: US 2013/0177916 A1
(43) Pub. Date: Jul. 11, 2013

(54) METHODS, DEVICES AND USES RELATED TO BIOFILMS

(71) Applicant: Hawley & Hazel Chemical Company (Zhongshan) Limited, Zhongshan (CN)

(72) Inventors: Wanghua CHEN, Zhongshan (CN); Xiaobin Zou, Zhongshan (CN)

(73) Assignee: Hawley & Hazel Chemical Company (Zhongshan) Limite, Zhongshan (CN)

(21) Appl. No.: 13/773,646

(22) Filed: Feb. 22, 2013

Related U.S. Application Data
(63) Continuation of application No. PCT/CN2011/001409, filed on Aug. 24, 2011.

(30) Foreign Application Priority Data
Aug. 24, 2010 (CN) 201010261075.7
Sep. 9, 2010 (CN) 201010276951.3

(51) Int. Cl. C12Q 1/02

(52) U.S. Cl. CPC C12Q 1/025
USPC 435/6.12; 435/289.1; 435/287.1; 435/252.1; 435/29

(57) ABSTRACT

The present disclosure provides a method of preparing a biofilm, comprising: inoculating an source bacteria sample to a substrate by directly dripping the source bacteria sample on the substrate, and culturing the source bacteria sample in a non-cyclic culture medium flow to form the biofilm sample on the substrate. The present disclosure also provides a device for the formation of a biofilm and uses of the biofilm in drug testing and screening. The device and method of the present disclosure saves culture time, reduces contamination, and can be used to form biofilm without anaerobic environment or pH adjustment in culture medium.

### CROSS REFERENCE TO RELATED APPLICATIONS

This is a continuation of PCT/CN2011/001409 filed on Aug. 24, 2011, which claims the priority to Chinese patent application No. 201010261075.7, filed on Aug. 24, 2010, and Chinese patent application No. 201010276951.3, filed on Sep. 9, 2010, all of which are incorporated herein by reference in their entirety.

### FIELD OF THE APPLICATION

The present disclosure relates to the field of biofilm, especially in relation to biofilm composition, formation, device and uses thereof.

### BACKGROUND

An in vitro model of a biofilm is useful for screening or testing potential drugs on their effect on inhibiting or preventing the formation of the biofilm, thereby predicating the drugs' effect on the biofilm formed in vivo as well as the effect of the drugs in treatment of diseases associated with the biofilm However, conventional methods of forming biofilms have many drawbacks, including for example, 1) complex in vitro structure that may not mimic or resemble biofilm in vivo; 2) high cost; 3) complex operation; 4) difficulty to control contamination; and 5) long experimental period.

Therefore, there are needs to continue to develop novel or improving devices and methods for forming biofilms in vitro that can overcome the above-mentioned shortcomings.

### SUMMARY

In one aspect of the present disclosure, a device for forming a biofilm is provided. The device includes: a base, a cover, a chamber defined by the base and the cover therebetween, a concave structure formed on a bottom surface of the chamber, and a first tube (or the first outlet) extending to the chamber and towards the concave structure, where the first tube connects the chamber and outside.

In certain embodiments, the first tube is integrated on the cover to reduce dead volume in the chamber, thus to reduce the possibility of contamination. In another embodiment, the first tube is integrated on the base.

In certain embodiments, the device further includes a flexible pipe, where the flexible pipe is connected to the first tube inside the chamber and extends to the bottom of the concave structure. The flexible pipe allows almost all fluids in the chamber to be drained oreluted outside the chamber through the flexible pipe and the first tube. Additionally, the flexible pipe permits an even mixing of a fluid introduced into the chamber through the first tube.

In certain embodiments, a fluid or a medium is introduced into the chamber through the first tube, optionally with the flexible pipe, and eluted outside the chamber through the same.

In certain embodiments, the concave structure has an arc surface.

In certain embodiments, the device further includes a position limiting structure and at least one substrate (or a plurality of substrates). The position limiting structure is located in the chamber to secure the position of the substrate or substrates in the chamber. The position limiting structure can be formed on opposite inner surfaces of the chamber. If the substrates are secured in the chamber, the substrate (or the substrates) would not float freely in the chamber when a fluid passes the chamber. In certain embodiments, the number of substrates can be 1, 2, 3, 4, 5, 6, 7, 8, 9, 10, 11, 12, 13, 14, 15, 16, 17, 18, 19, 20, 30, 40, 50 or more.

In certain embodiments, the device further includes a second tube (or the second outlet) extending into the chamber which connects the chamber and outside. In certain embodiments, the first tube can be used to introduce a fluid (or a medium) into the chamber and the second tube may elude the fluid outside the chamber. In the presence of a position limiting structure which is located or placed between the first and second tubes, the fluid is introduced into the chamber, passes through the concave structure, passes through the substrate or substrates secured or held stable in the chamber by the position limiting structure, and eludes outside the chamber through the second tube. The first and second tubes having openings within the chamber but are located at the two sides of the position limiting structures such that the fluid can pass through the substrate or substrates.

In certain embodiments, the chamber in the device further includes a second concave structure, wherein the second concave structure is corresponding to or directed towards the second tube. In certain embodiments, the second tube is connected to a second flexible pipe inside of the chamber and the pipe extends to the bottom of the second concave structure. The second flexible pipe allows almost all fluids in the chamber to be drained or eluted outside the chamber through the flexible pipe and the second tube as well.

In certain embodiments, the first and second tubes are both integrated to the cover; or both to the base. In certain embodiments, the first tube is integrated to the cover and the second tube is integrated to the base, or vice versa.

In certain embodiments, the base has a substantially flat top surface, and the concave structure is formed on the top surface of the base.

In another aspect of the present disclosure, a system for biofilm formation is provided. The system includes a first and a second devices having the structure disclosed herein. In certain embodiments, the system further comprises a culture medium source in fluid connection with the first and the second devices simultaneously. In certain embodiments, the system further comprises a first culture medium source in fluid connection with the first device and a second culture medium source in fluid connection with the second device.

In certain embodiments, the first device contains a first biofilm sample, and the second device contains a second biofilm sample which is different from the first sample.

In another aspect of the present disclosure, a system for testing the effect of drugs on biofilms is provided. The system includes: a first and a second devices having the herein-disclosed structures. In certain embodiments, the system further comprises a drug source in fluid connection with the first and the second devices simultaneously. In certain embodiments, the system further comprises a first drug source in fluid connection with the first device and a second drug source in fluid connection with the second device.

In another aspect of the present disclosure, a system for biofilm formation is provided. The system comprises a plurality of devices disclosed herein (e.g., a first device and a second device, a third device, a fourth device . . . and an N^{th} device (N≧2)). The system further comprises one culture medium source, two, three, four . . . or N culture medium sources in fluid connection with one or some or all device. For example, in one embodiment, the system comprises three devices and one culture medium source wherein the source is in fluid connection with all three devices. In another embodiment, the system comprises three devices and two culture medium sources wherein the one source is in fluid connection with two of the three devices and the other source is in fluid connection with the remaining one device. In another embodiment, the system comprises three device and three culture medium sources wherein the each source is in fluid connection with each of the three devices, respectively.

In another aspect of the present disclosure, a method of forming or preparing a biofilm in vitro is provided. The method includes the steps of: inoculating a source bacteria sample to at least one substrate, placing the substrate into the chamber of the device as disclosed herein wherein the substrate is secured to its position through the position limiting structure, tightening the base and the cover, and culturing the bacterial sample in the chamber to form a biofilm.

In certain embodiments, the substrate is pre-coated with a coating layer, which includes, for example, saliva protein, sterile saliva, mucin, adhesin, albumin or mucopolysaccharide.

In certain embodiments, the substrate may be a piece of human tooth, a piece of animal tooth, a hydroxyapatite substrate, a fluorapatite substrate, resin substrate, polyolefin substrate, polystyrene substrate, polyvinyl chloride substrate or polyurethane substrate, metal discs substrate, marble substrate or glass discs substrate.

In certain embodiments, source bacteria sample is gram-positive bacteria, gram-negative bacteria, aerobic bacteria or anaerobic bacteria, or a mixture thereof. In certain embodiments, the gram-positive bacteria is Enterococcus faecalis, Staphylococcus aureus, Staphylococcus epidermidis, Streptococcus viridans or any combination thereof. In certain embodiments, the gram-negative bacteria is Escherichia coli, Klebsiella pneumoniae, Proteus mirabilis, Pseudomonas aeruginosa and any combination thereof. In certain embodiments, the anaerobic bacteria is Streptococcus anginosus, Streptococcus australis, Streptococcus constellatus, Streptococcus mitis, Enterobacter sp, Actinomyces sp, Veillonella sp, Prevotella melaninogenica, Fusobacterium periodonticum or any combination thereof. In certain embodiments, aerobic bacteria is Aeromonas strain, Burkholderia strain, Flavobacterium strain, Microbacterium strain, Pseudomonas strain, Salmonella strain, Staphylococcus strain or any combination thereof. In certain embodiments, the source bacteria sample is oral bacteria sample selected from the group consisting of actinomyces viscosus, actinomyces naselundii, streptococcus mutans, streptococcus sanguis, streptococcus sobrinus, lactobacillus casei, lactobacillus acidophilius, candida albican, actinobacillus actinomycetemcomitans, veillonella parvula, fusobacterium nucleatum subsp. polymorphum, porphyromonas gingivalis, neisseria sp., and any combination thereof. In certain embodiments, the bacteria sample is an oral bacteria sample including saliva and/or dental bacteria or dental plaque or tongue coating. In certain embodiments, the saliva or dental plaque or tongue coating is collected from an animal (e.g., a cat or dog) or a human being.

In certain embodiments, the bacteria sample is cultured in the chamber by introducing a culture medium (a culture fluid) into the chamber through the first tube, passing the medium through the concave structure and the substrates which are secured or hold stable by the position limiting structure in the chamber, and eluding the medium out of the chamber through the second tube. In certain embodiments, the device in culturing may be place in the room temperature or about 37° C. In certain embodiments, the device in culture may be place regular culture environment not necessarily in absence of oxygen. In other words, the device in culture may not necessarily be in an anaerobic environment, the device can be place in an aerobic environment in the formation of biofilm. In certain embodiments, the pH of the culture medium needs not be adjusted or modulated.

In certain embodiments, the biofilm can be formed within about 8 hours, 12 hours, 24 hours, 36 hours, 48 hours, 60 hours, 72 hours, 4 days, 5 days, 6 days, 7 days in culture. In preferred embodiments, the biofilm can be formed within about 8-36 hours of culture. In certain embodiments, the biofilm can be formed within about 24 hours of culture with the device in an aerobic environment, at a temperature about 37° C.

In anther aspect of the present disclosure, an in vitro composition of biofilm is provided. The biofilm composition comprises: a substrate, a biofilm of formed on the substrate, where the biofilm is formed by the formation methods and device as disclosed herein.

In certain embodiments, the composition further comprises a coating layer between the substrate and the biofilm. In certain embodiments, the biofilm comprises a mixture of anaerobic and aerobic bacteria. In certain embodiments, the anaerobic bacteria is Streptococcus anginosus, Streptococcus australis, Streptococcus constellatus, Streptococcus mitis, Enterobacter sp, Actinomyces sp, Veillonella sp, Prevotella melaninogenica, Fusobacterium periodonticum or any combination thereof. In certain embodiments, aerobic bacteria is Aeromonas strain, Burkholderia strain, Flavobacterium strain, Microbacterium strain, Pseudomonas strain, Salmonella strain, Staphylococcus strain or any combination thereof.

In another aspect of the present disclosure, methods of using the biofilms formed herein are provided. For example, the biofilm can be used in a method of screening drug candidate or testing the effect of a drug on inhibiting or preventing formation of biofilm is provided. The method includes the steps of contacting the biofilm with an agent (or a drug candidate), culturing the biofilm using the culture medium in the presence of the agent or in the absence of the agent, and analyzing the biofilm.

In another aspect of the present disclosure, a method for testing the effect of a first drug and a second drug which is different from the first drug on inhibiting or preventing formation of oral bacteria biofilm is provided. The method includes: contacting a first biofilm with a first agent in a first device, contacting a second biofilm with a second agent in a second device, culturing the first and second device, and analyzing the first biofilm and the second biofilm after the culture.

In another aspect of the present disclosure, the biofilms formed herein can also be used to test whether the biofilms themselves can be used for water treatment or pollution removal or oil treatment. The methods comprise the step of contacting a liquid to be treated with a biofilm as disclosed herein and analyzing the liquid after having been contacted.

### BRIEF DESCRIPTION OF DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a sectional view of a device for forming a biofilm sample of an embodiment of the present disclosure.

FIG. 1a illustrates a sectional view of a device for forming a biofilm sample of another embodiment of the present disclosure.

FIG. 1b illustrates a sectional view of a device for forming a biofilm sample of another embodiment of the present disclosure.

FIG. 2 illustrates a top view of the base of the device in FIG. 1.

FIG. 3 illustrates a schematic diagram of a parallel system for testing drugs on the effect on dental plaque.

FIG. 4a illustrates a top view of rack for holding devices for forming biofilms.

FIG. 4b shows a cross sectional view of the rack of FIG. 3.

FIG. 5 illustrates a flow chart of a method for testing drugs on the effect on dental plaque.

FIG. 6a and FIG. 6b are schematic diagrams of in vitro model of dental plaque.

FIG. 7 shows optical density and number of the bacteria colonies of a dental plaque at different culture time.

FIG. 8 shows optical density and pH value of a bacteria liquid in a device at different culture time.

FIG. 9 shows a fluorescent image of a dental plaque on a hydroxyapatite substrate.

FIG. 10 shows a fluorescent image of a dental plaque homogenate sample.

FIG. 11 shows the results of a PCR-DGGE analysis.

FIG. 12a, FIG. 12b and FIG. 12c show SEM images of the dental plaque on the hydroxyapatite substrate.

FIG. 13 shows a histogram illustrating the optical density of a dental plaque sample treated with a test toothpaste.

FIG. 14 shows a histogram showing the optical density of three dental plaque samples treated with two test mouthwashes and water, respectively.

### DETAILED DESCRIPTION OF THE INVENTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

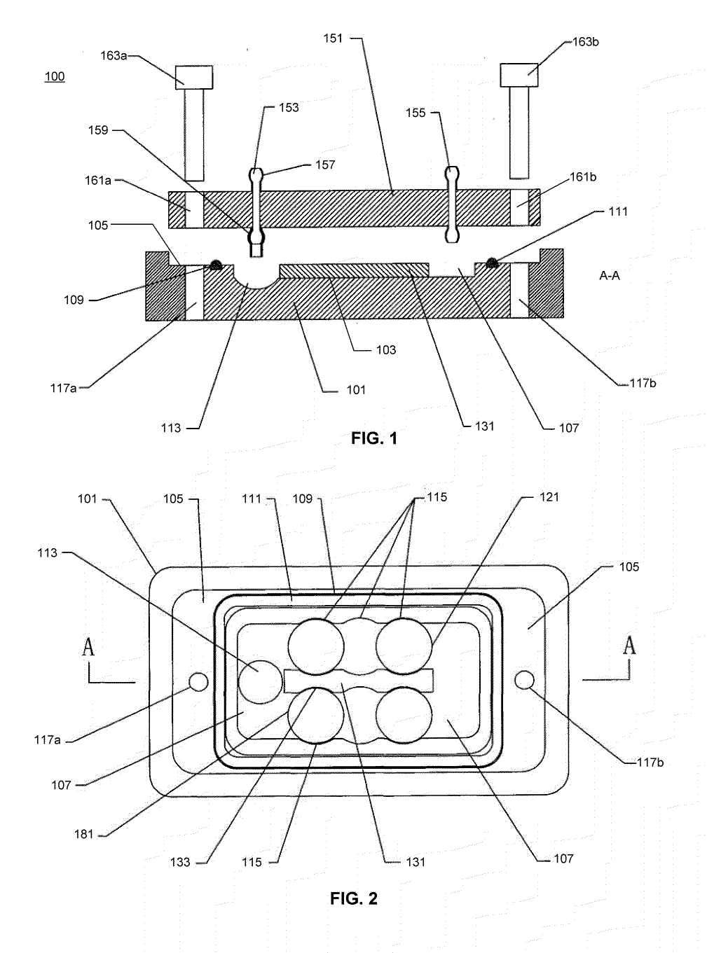

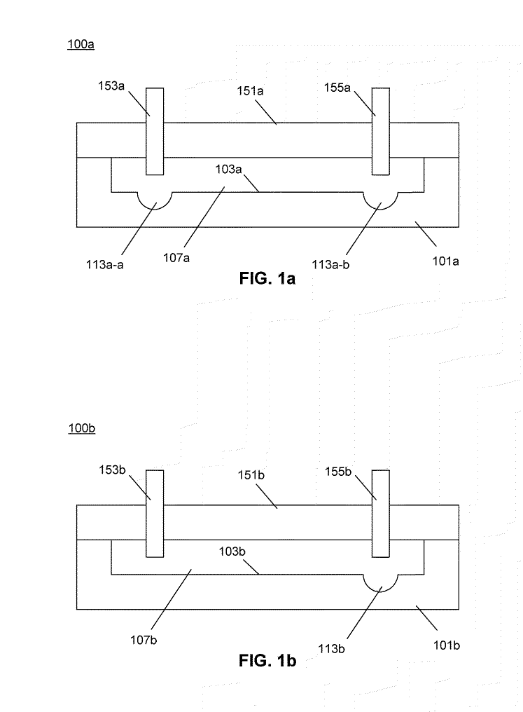

One aspect of the present disclosure pertains to a device for the in vitro formation of a biofilm. FIG. 1 illustrates a section view of a device 100 for the in vitro formation of a biofilm. The device 100 includes a base 101 and a cover 151. The base 101 has a substantially flat top surface 103, and a wall 105 is raised from the top surface 103. A chamber 107 is defined by the wall 105, the top surface 103, and a bottom surface of the cover 151.

In an alternative embodiment, the chamber 107 may be defined by the bottom surface of the cover 151, a wall raised from the bottom surface of the cover 151, and the top surface 103 of the base 101. In another alternative embodiment, the chamber 107 may be defined by the bottom surface of the cover 151, a wall raised from the bottom surface of the cover 151, the wall 105, and the top surface 103 of the base 101.

The wall 105 has a substantially flat top surface on which a groove 109 is formed, and the chamber 107 is within the groove 109. The groove 109 receives therein a sealing member 111 (e.g., rubber seal ring) which seals the chamber 107 when the cover 151 and the base 101 are attached to each other tightly so as to prevent liquid or fluid from leaking out of the chamber. The sealing member surrounds the chamber 107 and is located at the outskirt of the chamber 107.

It will be appreciated by those skills in the art that in an alternative embodiment, the groove 109 may be formed on the bottom surface of the cover 151.

The base 101 further includes a concave structure 113 formed on the top surface 103 of the base 101 for collecting fluids. In addition, two threaded holes 117a and 117b extend from the top surface of the wall 103 substantially vertically at opposite ends of the base 101, respectively.

The cover 151 includes a first tube 153 (or first port) and a second tube 155 (or second port) integrated thereon. The first tube 153 and the second tube 155 are located at opposite ends of the cover 151 but within the chamber 107 and extend beyond the top and bottom surfaces of the cover 151, and the first tube 153 extends to the concave structure 113 when the cover 151 is attached to the base 101. Preferably, the first tube 153 and the second tube 155 have bulged structures 157 near their ends, such that flexible pipes can be fit on them tightly sealed.

Those skills in the art will appreciate that alternatively the first and the second tubes 153 and 155 may be integrated on the base 101. It will be appreciated by those skills in the art that in an alternative embodiment, instead of integrated on the cover 151, the first tube 153 and the second tube 155 may be made independently. In other words, one may be mounted on the cover 151 and the other on the base 101.

The cover 151 further includes two through holes 161a and 161b at opposite ends, which through holes correspond to the threaded holes 117a and 117b formed on the base 101, thus the base 101 and the cover 151 can be attached to each other tightly using bolts 163a and 163b.

In order to drain the fluids from the chamber 107 thoroughly, a piece of flexible pipe 159 is fit on the lower end of the first tube 153 such that the flexible pipe 159 extends to the bottom of the concave structure 113 substantially. In one embodiment, the flexible pipe 159 may be made of rubbers and silica gels, preferably silica gels. The length of the flexible pipe is such that once the cover 151 and the based 101 are tightened by bolts 163 a and 163b the pipe would touch the bottom of the concave structure 113, or even bend a bit, and would be able to remove or draw out thoroughly liquid or fluid in the chamber as well as in the concave structure. Therefore, the fluid in the chamber may be drained thoroughly through the flexible tube 159.

FIG. 2 illustrates a top view of the base 101. A plurality of sunken arc surfaces 115 and 133 are formed on the inner surface of the chamber 107 and on a position limiting bar 131, respectively. The sunken arc surfaces 115 and 133 can work together to secure disk shaped substrates 121 in their own position stably and prevent them from moving or float freely in the chamber 107.

In certain embodiments, the position limiting bar 131 may be separate from the base 101. In another embodiment, the position limiting bar 131 may be integrated on the base 101

In certain embodiments, the substrates 121 and the position limiting bar 131 across the passage between the first tube 153 and the second tube 155, thus the flow from the first tube 153 to the second tube 155 is forced to pass through the top surface of the substrates 121.

In certain embodiments, the base 101 and the cover 151 are made of high temperature endurable and corrosion-resistant materials. Examples of such materials include but not limited to polymethylmethacrylate (PMMA), polycarbonate (PC), polypropylene (PP), polystyrene (PS), polyvinyl chloride (PVC), and polyamide (PA). The materials can be sterilized at high temperature to create a sterilized condition the chamber 107 before the test. Any other high temperature endurable and corrosion resistant metals can also be used.

The volume and the size of the chamber 107 may be determined according to specific applications. For example, the volume of the chamber107 may be 5 ml, 6 ml, 7 ml, 8 ml, 9 ml, 10 ml, 15 ml, 50 ml etc.

FIG. 1a illustrates a section view of a device 100a for forming a biofilm sample of another embodiment of the present disclosure. The device 100a includes a base 101a and a cover 151a. A chamber 107a is defined by the base 101a and a cover 151a therebetween. A first concave structure 113a-a and a second concave structure 113a-b are formed on a substantially flat top surface 103a of the base 101a. A first tube 153a and a second tube 155a are integrated on the cover 151a for introducing fluids into the chamber 107a and/or draining fluids out the chamber 107a. The first tube 153a and the second tube 155a extend to the first concave structure 113a-a and the second concave structure 113a-b, respectively.

FIG. 1b illustrates a section view of a device 100b for forming a biofilm sample of another embodiment of the present disclosure. The device 100b includes a base 101b and a cover 151b. A chamber 107b is defined by the base 101b and a cover 151b therebetween. A concave structure 113b is formed on the right hand of a substantially flat top surface 103b of the base 101b. A first tube 153b and a second tube 155b are integrated on the cover 151b for introducing fluids into the chamber 107b and/or draining fluids out the chamber 107b. The second tube 155b extends to the second concave structure 113b.

In an alternative embodiment, a first tube, which connects a chamber of a device for forming a biofilm sample, extends to a fluid collecting area of the chamber, which area a fluid in the chamber will flow to when the fluid needs to be drained out. Therefore, the fluids in the chamber may be drained out more thoroughly. For example, the first tube may extend to a corner of the chamber, and when the fluids in the chamber need to be drained out, the device may be inclined such that the fluids flow to the corner.

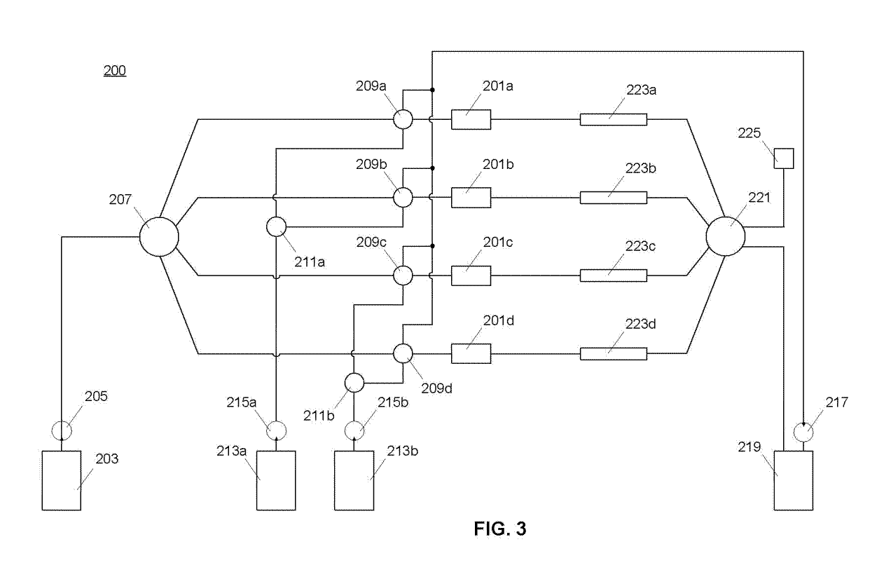

FIG. 3 illustrates a schematic diagram of a parallel system 200 for biofilm formation and drug test. The system 200 includes devices 201a, 201b, 201c, and 201d. Each of the devices has the same structure with that of the device 100.

The devices 201a, 201b, 201c, and 201d are in fluid communication with a culture medium source 203. A pump 205 is connected to the culture medium source 203 to pump culture medium from the culture medium source 203 to a manifold 207. A culture medium source may be a device which provides a culture medium. For example, a culture medium source may be a tank containing a culture medium. The manifold 207 has four output ports connected to valves 209a, 209b, 209c, and 209d, respectively.

Each of the valves 209a, 209b, 209c, and 209d has three positions. When the valve 209a is switched to a first position, it connects the manifold 207 and a first port of the device 201a, such that the culture medium from the culture medium source 203 may be transported into the device 201a; when the valve 209a is switched to a second position, it connects a manifold 211a and the first port of the device 201a, such that drug from a first drug source 213a may be pumped by a pump 215a to the manifold 211a and then transported into the device 201a; when the valve 209a is switched to a third position, it connects the first port of the device 201a and a pump 217, such that the fluids in the device 201a may be drained into a waste liquid collector 219. Because there is a concave structure formed in each device and the first port of each device extends to the bottom of the concave structure, the fluids in each device will be collected in the concave structure and will be drained through the first port thoroughly. A drug source may be a device which provides a drug solution or a solution of a mixture of drugs. For example, a drug source may be a tank containing a drug solution or a solution of a mixture of drugs.

When the valve 209b is switched to a first position, it connects the manifold 207 and a first port of the device 201b, such that the culture medium from the culture medium source 203 may be transported into the device 201b; when the valve 209b is switched to a second position, it connects the manifold 211a and the first port of the device 201b, such that drug from the first drug source 213a may be pumped by the pump 215a to the manifold 211a and then transported into the device 201b; when the valve 209b is switched to a third position, it connects the first port of the device 201b and the pump 217, such that the fluids in the device 201b may be drained into the waste liquid collector 219.

When the valve 209c is switched to a first position, it connects the manifold 207 and a first port of the device 201c, such that the culture medium from the culture medium source 203 may be transported into the device 201c; when the valve 209c is switched to a second position, it connects a manifold 211b and the first port of the device 201c, such that drug from a second drug source 213b may be pumped by a pump 215b to the manifold 211b and then transported into the device 201c; when the valve 209c is switched to a third position, it connects the first port of the device 201c and the pump 217, such that the fluids in the device 201c may be drained into the waste liquid collector 219.

When the valve 209d is switched to a first position, it connects the manifold 207 and a first port of the device 201d, such that the culture medium from the culture medium source 203 may be transported into the device 201d; when the valve 209c is switched to a second position, it connects the manifold 211b and the first port of the device 201d, such that drug from the second drug source 213b may be pumped by the pump 215b to the manifold 211b and then transported into the device 201d; when the valve 209c is switched to a third position, it connects the first port of the device 201d and the pump 217, such that the fluids in the device 201d may be drained into the waste liquid collector 219.

In certain embodiments, the ports of the valves 209a˜209d that connect to the pump 217 may be connected to four different pumps, respectively, instead, such that the fluids in the devices 201a˜201d may be drained out separately. In certain embodiments, the ports of the valves 209a˜209d that connect to the pump 217 may be connected to four inlet ports of a dispensing pump, respectively, instead.

In certain embodiments, a dispensing pump may be used to replace the combination of the pump 205 and the manifold 207. For example, a dispensing pump from ISMATEC may be used. For example, the dispensing pump model number IPC24 may be used for a 24 channel system.

Similarly, in certain embodiments, a dispensing pump may be used to replace the combination of the pump 215a and the manifold 211a, and a dispensing pump may be used to replace the combination of the pump 215b and the manifold 211b.

In certain embodiments, the drug from the first drug source 213a is different from that from the second drug source 213b.

Each of the devices 201a, 201b, 201c, and 201d has a second port. The second ports of the devices 201a, 201b, 201c, and 201d are connected to a valve 221 through flow resistors 223a, 223b, 223c, and 223d, respectively. Because there is a flow resistor in each channel which flow resistor has a large flow resistance such that the flow resistance of the rest part of the channel may be neglected, and the flow rate of each channel may be adjusted precisely. For example, the flow resistors 223a, 223b, 223c, and 223d have the same flow resistance, when the valves 209a, 209b, 209c, and 209d are switched to the first position, the four parallel channels will have substantially the same flow rate. A flow resistor may be a pipe having a very small inner diameter, for example, capillary. In this case, the flow resistance of a flow resistor may be adjusted by adjust the length of the pipe.

The valve 221 has five positions. When the valve 221 is switched to a first position, it connects the second port of the device 201a and a sampler 225, and meanwhile it connects the second ports of the devices 201b, 201c, and 201d and the waste liquid collector 219. The sampler 225 is to sample the liquid from the devices to monitor the conditions therein.

When the valve 221 is switched to a second position, it connects the second port of the device 201b and the sampler 225, and meanwhile it connects the second ports of the devices 201a, 201c, and 201d and the waste liquid collector 219.

When the valve 221 is switched to a third position, it connects the second port of the device 201c and the sampler 225, and meanwhile it connects the second ports of the devices 201a, 201b, and 201d and the waste liquid collector 219.

When the valve 221 is switched to a fourth position, it connects the second port of the device 201d and the sampler 225, and meanwhile it connects the second ports of the devices 201a, 201b, and 201c and the waste liquid collector 219.

When the valve 221 is switched to a fifth position, it connects the second ports of the devices 201a, 201b, 201c, and 201d and the waste liquid collector 219. In this position, the sampler does not sample the fluids from the devices, and the fluids are all drained into the waste liquid collector 219.

Those skills in the art will appreciate that under the teaching of the present disclosure, many modifications may be made to the arrangement of the system 200. For example, the system 200 may have a plurality of devices (4, 5, 6, 7, 8, 9, 10, . . . N), culture medium source, drug sources, flow resistors, and so on.

In certain embodiments, the system 200 may be controlled by a computer system, thus an experiment may be conducted automatically.

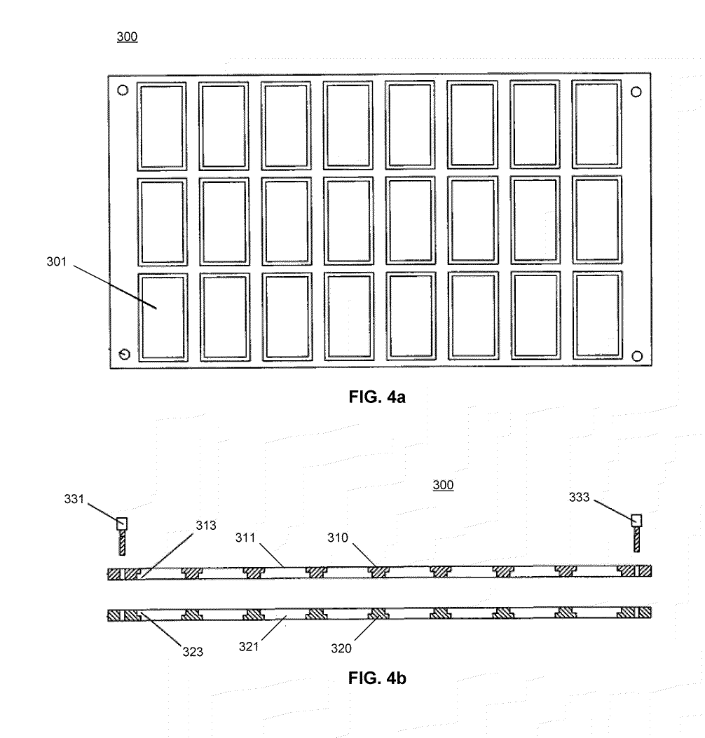

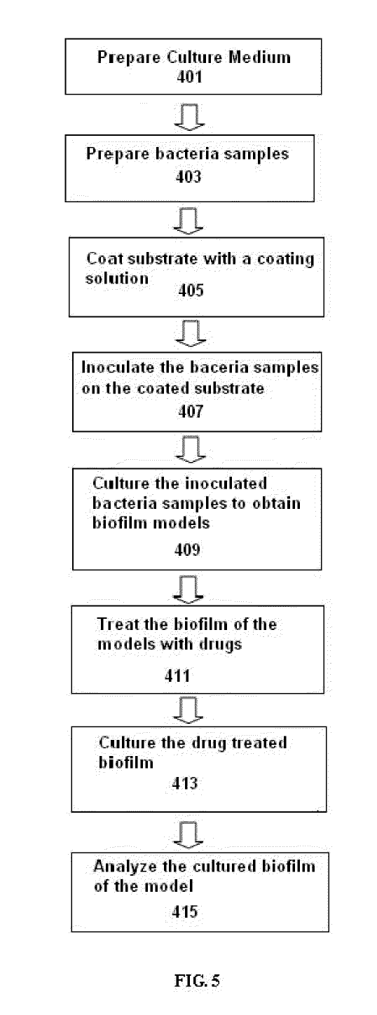

Referring to FIG. 4a and FIG. 4b, a plurality of devices 301 may be mounted on a rack 300. Each device 301 may be the device 100 described above. The rack 300 includes an upper frame 310 and a lower frame 320. The upper frame 310 has a plurality of upper openings 311 smaller than the devices 301 extending through the top surface of the upper frame 310. The upper frame 310 further has a plurality of lower openings 313 which the devices 301 may fit in, extending from the upper openings 311 through the bottom surface of the upper frame 310.

The lower frame 320 has a plurality of lower openings 321 smaller than the devices 301 extending through the bottom surface of the lower frame 320. The lower frame 320 further has a plurality of upper openings 323 which the devices 301 may fit in, extending from the lower openings 321 through the top surface of the lower frame 320. Therefore, a plurality of devices 301 may be sandwiched by the upper frame 310 and the lower frame 320, and be fixed in the lower openings 313 of the upper frame 310 and the upper openings 323 of the lower frame 310.

Another aspect of the present disclosure provides methods to form a biofilm by using the device 100 or a plurality of biofilms by using system 200. As illustrated in FIG. 5, in 401, a culture medium is prepared. The method of preparing the culture medium is well known in the art. In certain embodiments, the culture medium is prepared according to Sissons, et al. J. Dent Res. 1991; 70(11): 1409-16. The culture medium can be any culture medium that can support the growth of oral bacterial samples. An example of the culture medium includes but not limited to the basic mucin medium (the “BMM”).

In 403, a bacteria sample is prepared. The bacteria sample may be any bacteria involved in biofilm formation, including without limitation to gram-positive bacteria, gram-negative bacteria, aerobic bacteria or anaerobic bacteria or any combination thereof.

In certain embodiments, the gram-positive bacteria is Enterococcus faecalis, Staphylococcus aureus, Staphylococcus epidermidis, Streptococcus viridans or any combination thereof. In certain embodiments, the gram-negative bacteria is Escherichia coli, Klebsiella pneumoniae, Proteus mirabilis, Pseudomonas aeruginosa and any combination thereof. In certain embodiments, the anaerobic bacteria is Streptococcus anginosus, Streptococcus australis, Streptococcus constellatus, Streptococcus mitis, Enterobacter sp, Actinomyces sp, Veillonella sp, Prevotella melaninogenica, Fusobacterium periodonticum or any combination thereof. In certain embodiments, aerobic bacteria is Aeromonas strain, Burkholderia strain, Flavobacterium strain, Microbacterium strain, Pseudomonas strain, Salmonella strain, Staphylococcus strain or any combination thereof.

In certain embodiments, the bacteria sample is an oral bacteria sample. For example, the oral bacteria sample is a dental plaque from a human being or an animal (e.g., a cat, a dog, a cow, a mouse, and so on). In certain embodiments, the bacteria sample is oral bacteria including without limitation to Actinomyces viscosus, Actinomyces naselundii, Streptococcus mutans, Streptococcus sanguis, Streptococcus sobrinus, Lactobacillus casei, Lactobacillus acidophilius, Candida albican, Actinobacillus actinomycetemcomitans, Veillonella parvula, Fusobacterium nucleatum subsp. polymorphum, Porphyromonas gingivalis, Neisseria sp., and any combination thereof.

In certain embodiments, the bacteria sample is a saliva sample. The saliva sample is collected from a human being or an animal. In another embodiment, the saliva sample is diluted with the culture medium prepared in 401 and sucrose.

Optionally, in 405, a substrate 121 is pre-coated with a coating solution to form a coating layer. Examples of coating solution include but not limited to saliva protein, sterile saliva, artificial saliva, mucin, adhesin, albumin or mucopolysaccharide. In certain embodiments, the coating solution is sterile saliva. The coating solution may help to attach or anchor free-floating, planktonic bacteria onto a surface of a substrate 121, which stimulates the formation of biofilm in vivo. In addition, the coating solution facilitates the growth of the bacteria sample by attaching the bacteria on the surface of the substrate.

The substrate described herein can be any carrier with a surface that can support the growth of the bacteria sample. In certain embodiments, the substrate is resin substrate, polyolefin substrate, polystyrene substrate, polyvinyl chloride substrate, polyurethane substrate, metal discs substrate, marble substrate or glass discs substrate. In certain embodiments, the substrate is made of material that has chemical components and a structure similar to teeth. Examples of substrate includes but not limited to hydroxyapatite substrate, the fluorapatite substrate or a tooth slice of an animal e.g. dogs, cattle and pigs, or a tooth slice of a human being. In certain embodiments, the substrate is a hydroxyapatite substrate. The shape and the size of the substrate can be determined according to the practical need as far as the shape and size correspond to the position limiting structure 131 and the inside rim of the chamber 107 in the device disclosed herein so that the substrate can be secured or held stably in the chamber in the presence of fluid or culture medium passing through the chamber 107. In certain embodiments, the shape of the substrate may be disk or rectangle wafer. In certain embodiments, the length of the substrate may be about 3˜25 mm and the thickness may be about 0.5 mm. In certain embodiments, the diameter of the substrate may be about 3˜25 mm and the thickness may be about 0.5 mm.

In 407, the substrate 121 coated with the coating layer is inoculated with the bacteria sample prepared in 403. The coated substrate 121 is then put into and secured by the sunken arc surfaces 115 and 133 in the chamber 107. The bacteria sample is inoculated onto the coated substrate 121 by adding the bacteria sample into the chamber 107 until the substrate 121 is immersed in the bacteria sample. In certain embodiments, the bacteria sample may be inoculated onto the coated substrate 121 using a transferpettor. After the substrate 121 is loaded on the base 101 of a device 100 and inoculated with the bacteria sample, the cover 151 of the device 100 is attached to the base 101 tightly to provide a sealed chamber.

In certain embodiments, two or more devices may be used to for a plurality of biofilms (different or same biofilms) and test same drugs for different biofilms or different drugs for the same biofilms. In certain embodiments, the system 200 is used. The devices 201a and 201b are loaded with substrates inoculated with a first bacteria sample, and the devices 201c and 201d are loaded with substrates inoculated with a second bacteria sample which is different from the first bacteria sample. The devices may be water-bathed under about 37° C.

In 409, the bacteria sample is cultured for a period of time using the culture medium to allow the bacteria sample to form biofilm on the substrate 121. In certain embodiments, the bacteria sample is cultured in the chamber 107 by introducing a culture medium into the chamber 107 through the tube 153, passing the culture medium through the concave structure 113 and the substrate 121 in the chamber 107, eluting the medium outside of the chamber through the tube 155 (e.g., into waste liquid collector 219). The culture medium flowing through the chamber 107 is eluted outside of the chamber 107 and collected to be discarded without flowing into the chamber again. In other words, the culture medium flow is non-cyclic. The bacteria sample is cultured in a continuous fresh culture medium flow. The culture medium flow transported and diluted the acid substance produced by the bacteria out of the chamber 107 in formation of the biofilm, thereby maintaining the culture condition in the chamber 107 around 7.0. In certain embodiments, the device 100 is water-bathed at about 37° C. or placed at room temperature. In certain embodiments, the bacteria sample is cultured without the necessity of injecting anaerobic gas to create anaerobic environment. In certain embodiments, the device 100 can be placed in an aerobic environment in the formation of biofilm. In certain embodiments, dilution rate of the culture medium may be set to 0.5˜5 h⁻¹. In certain embodiments, the dilution rate is set to 0.1 h⁻¹, 0.2 h⁻¹, 0.3 h⁻¹, 0.4 h⁻¹, 0.5 h⁻¹, 0.6 h⁻¹ 0.7 h⁻¹ 0.8 h⁻¹ or 0.9 h⁻¹. The dilution rate described herein refers to the ratio of the volume of the culture medium that is injected into the chamber to the volume of the chamber per hour. In certain embodiments, the biofilm can be formed within about 8 hours, 12 hours, 24 hours, 36 hours, 48 hours, 60 hours, 72 hours, 4 days, 5 days, 6 days, 7 days in culture. In preferred embodiments, the biofilm can be formed within about 8-36 hours in culture. In certain embodiments, the biofilm can be formed within about 24 hours of culture with the device 100 in an aerobic environment, at a temperature about 37° C. In certain embodiments, the system 200 is used. The valves 209a, 209b, 209c and 209d are switched to the first position to transport the culture medium into the devices 201a, 201b, 201c and 201d, passing the culture medium through the concave structures and the substrates in the devices, venting through the tube into waste liquid collector 219.

Another aspect of the present disclosure relates to the use of the biofilm formed in the device or devices disclosed herein. As shown in FIG. 5, in 411, the culture medium in the chamber 107 of the device 100 is drained out by the first tube 153, and optionally together with the flexible pipe 159. The concave structure 113 collects the culture medium in the chamber 107, thereby facilitating the drain of the culture medium out of the chamber 107. Then a drug solution containing a drug (or an agent, or drugs, or agents) is injected into the chamber 107 until the biofilm on the substrate 121 in the chamber 107 is immersed in the drug solution. In certain embodiments, the biofilm is immersed in the drug solution for 30 seconds, about 1 minute, 2 minutes, 3 minutes, 4 minutes, 5 minutes, 6 minutes, 7 minutes, 8 minutes, 9 minutes, 10 minutes, 20 minutes, 30 minutes, 60 minutes, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days and so on. In certain embodiments, the system 200 is used. The valves 209a˜209d are switched to the third position to drain the liquid in the device 201a, 201b, 201c and 201d into the waste liquid collector 219. Then the valves 209a˜209d are switched to the second position to transport the first drug solution contain a first drug into the devices 201a and 201b and the second drug solution containing a second drug into the devices 201c and 201d such that the biofilm is immersed in the drugs. In certain embodiments, the first drug is different from the second drug. In another embodiment, the first drug is same as the second drug.

In 413, the drug solution in the chamber 107 of the device 100 is drained out by the first tube 153, and optionally together with the flexible pipe 159. The concave structure 113 collects the drug in the chamber 107, thereby facilitating the drain of the drug solution out of the chamber 107. Then the biofilm is cultured in the chamber 107 by introducing a culture medium into the chamber 107 through the tube 153, passing the culture medium through the concave structure 113 and the substrate 121, eluting through the tube 155 outside the chamber 107 (e.g., into waste liquid collector 219). In certain embodiments, the device 100 is water-bathed at about 37° C. or placed at room temperature. In certain embodiments, the biofilm is cultured without injecting anaerobic gas for creating anaerobic environment. In certain embodiments, dilution rate of the culture medium may be set to 0.5˜5 h⁻¹. In certain embodiments, the dilution rate is set to 0.1 h⁻¹, 0.2 h⁻¹, 0.3 h⁻¹, 0.4 h⁻¹, 0.5 h⁻¹, 0.6 h⁻¹ 0.7 h⁻¹ 0.8 h⁻¹ or 0.9 h⁻¹. In certain embodiments, the biofilm is cultured for about 8 hours, 12 hours, 16 hours, 24 hours, 36 hours, 48 hours, 60 hours, 72 hours, 4 days, 5 days, 6 days, 7 days. In certain embodiments, the biofilm is cultured for 16 hours in an aerobic environment, at a temperature about 37° C. In certain embodiment, the system 200 is used. The valves 209a˜209d are switched to the first position to transport the culture medium into the devices 201a˜201d, passing the culture medium through the concave structures and the substrates in the devices, eluting through the tube into waste liquid collector 219.

In 415, the biofilms are analyzed. The base 101 is separated from the cover 151. The substrate 121 is taken out from the chamber 107 and ultrasonic vibrated and/or shaked in a suspension liquid to obtain a suspension of the biofilm for analysis. In certain embodiments, the biofilm on the substrate is analyzed directly. The analysis method includes but not limited to spectrophotometry, fluorescent microscope and scanning electron microscope (SEM). In certain embodiments, spectrophotometry is used to analyze the optical density of the biofilm, thereby determining the amount of the biofilm. In certain embodiments, fluorescent microscope is used to determine the activity of the biofilm. In certain embodiments, SEM is used to determine the space structure of the biofilm on the substrate 121.

In another aspect of the present disclosure, the biofilms formed herein can also be used to test whether the biofilms themselves can be used for water treatment or pollution removal or oil treatment. The biofilm is formed on the substrate 121 using the method and the device described herein. The culture medium is drained out from the chamber 107 by using the first tube 153. A liquid to be test is injected into the chamber 107 of the device 100 through the tube 153, passes through the concave structure 113 and the biofilm on the substrate 121, elutes through the tube 155. The eluted liquid is collected and analyzed in comparison to the liquid before the injection into the chamber. The flow speed of the test liquid can adjusted according to the practical need. In certain embodiments, the flow speed is 0.1˜5 h⁻¹. In certain embodiments, the liquid to be test is waste water containing organic substances. The biofilm decomposed the organic substances when the waste water flows over the biofilm on the substrates 121 in the chamber 107 of device 100. The eluted liquid is analyzed by testing the content of organic substances therein. In certain embodiments, the thickness of the biofilm is measured for analysis. In certain embodiments, the liquid to be test is liquid with oil. The biofilm absorbs the oil when the test liquid flows over the biofilm on the substrates 121 in the chamber 107 of device 100. The eluted liquid is analyzed by testing the content of the oil in it. In certain embodiments, the content of the oil absorbed on the biofilm is measured for the analysis.

Another aspect of the present disclosure relates to biofilms formed in devices or device disclosed herein. A “biofilm” used herein refers to an aggregation of microorganisms that grow on a substrate and interact with each other. The aggregated microorganisms are usually embedded within a self-produced extracellular polymer matrix substance which is known to be a polymeric conglomeration generally composed of extracellular DNA, proteins, and polysaccharides. Biofilms may form on living or non-living surfaces and can be prevalent in natural, industrial and hospital settings. The microorganisms growing in a biofilm are physiologically distinct from planktonic, free-floating cells of the same organism, as the environment of the biofilm allows them to cooperate and interact in various ways in the matrix. One benefit of this environment is an increased resistance to detergents and antibiotics, as the dense extracellular matrix and the outer layer of microorganisms protect the interior of the community.

Without being bound to any theories, formation of a biofilm begins with the attachment of initial free-floating microorganisms to a substrate. If the first colonists adhering to the surface are not immediately separated from the surface, they may anchor themselves more permanently using cell adhesion molecules. The first colonists facilitate the arrival of other cells by providing more diverse adhesion sites and beginning to build the matrix that holds the community of microorganisms together. Some species are able to attach to a surface on their own. Others are often able to anchor themselves to the matrix or directly to earlier colonists. Once colonization has begun, the biofilm grows through a combination of cell division and/or recruitment. The biofilm causes cause a number of chronic infections and diseases including without limitation to atherosclerosis, chronic sinusitis, cystic fibrosis, endocarditis, inner ear infections, leptospirosis, osteomyelitis, periodontal diseases and urinary tract infections.

The microorganisms in the biofilm include a mixture of bacteria, for example, including gram-positive, gram-negative bacteria, anaerobic bacteria, aerobic bacteria, and any combination thereof. The gram-positive bacteria includes without limitation to Enterococcus faecalis, Staphylococcus aureus, Staphylococcus epidermidis, Streptococcus viridans or any combination thereof. The gram-negative bacteria includes without limitation to Escherichia coli, Klebsiella pneumoniae, Proteus mirabilis, Pseudomonas aeruginosa and any combination thereof. Bacteria may be aerobic or anaerobic. the anaerobic bacteria is Streptococcus anginosus, Streptococcus australis, Streptococcus constellatus, Streptococcus mitis, Enterobacter sp, Actinomyces sp, Veillonella sp, Prevotella melaninogenica, Fusobacterium periodonticum or any combination thereof. The aerobic bacteria includes without limitation to Aeromonas strain, Burkholderia strain, Flavobacterium strain, Microbacterium strain, Pseudomonas aeruginosa, Salmonella strain, Staphylococcus strain or any combination thereof. In certain embodiments, the biofilm includes oral bacteria. The oral bacteria includes without limitation to Actinomyces viscosus, Actinomyces naselundii, Streptococcus mutans, Streptococcus sanguis, Streptococcus sobrinus, Lactobacillus casei, Lactobacillus acidophilius, Candida albican, Actinobacillus actinomycetemcomitans, Veillonella parvula, Fusobacterium nucleatum subsp. polymorphum, Porphyromonas gingivalis, Neisseria sp., and any combination thereof.

In certain embodiments, the biofilm composition disclosed herein are formed by inoculating saliva onto a substrate (or a pre-coated substrate) and culturing in the conditions disclosed herein.

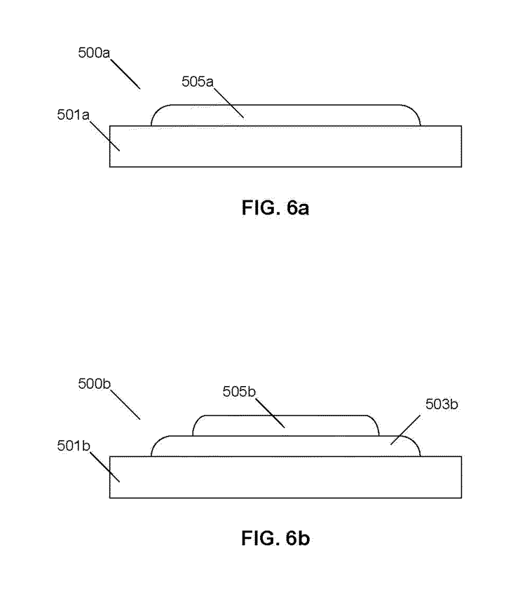

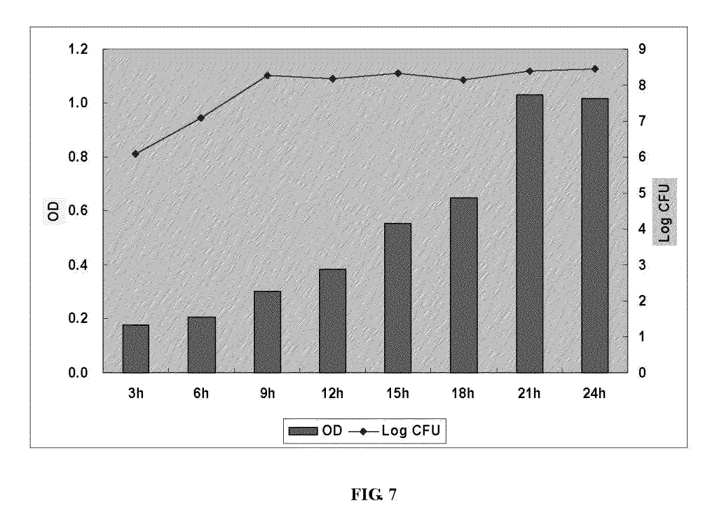

The present disclosure also provides an in vitro composition of biofilm that is prepared by the method described above. As illustrated in FIG. 6a, an composition of biofilm 500a includes a substrate 501a and a biofilm 505a formed on the substrate 501a.

As illustrated in FIG. 6b, an in vitro composition of biofilm 500b includes a substrate 501b, a coating layer 503b on the substrate 501b, and a biofilm 505b on the coating layer 503b. The coating layer 503b can be formed using the coating solution described above.

The present device, method, and uses disclosed herein present many unexpected advantages. For example, the structure of the device 100 is very simple with only two main parts, the cover 151 and the base 101 in defining the chamber 107, which significantly reduces the difficulty, the procedure and the cost in manufacturing the device 100. Meanwhile, the materials of the device 100 are selected from the group consisting of polymethylmethacrylate (PMMA), polycarbonate (PC), polypropylene (PP), polystyrene (PS), polyvinyl chloride (PVC), polyamide (PA) and a corrosion-resistant metal, which can be sterilized at high temperature, thereby guaranteeing the sterilized condition in the chamber 107 before the test.

The fluids in the chamber 107 may be drained more thoroughly through the flexible tube 159, reducing the volume of the culture medium or the drug left in the chamber 107 when they are drained out, thereby reducing the effect on the test caused by the culture medium or the drug left in the chamber 107. In addition, because there is a concave structure 113 formed in each device 100 and the first port of each device 100 extends to the bottom of the concave structure 113, the fluids in each device 110 will be collected in the concave structure 113 and will be drained through the first port thoroughly, which also reduces the effect on the test caused by the culture medium or the drug left in the chamber 107.

The pH value of the culture condition is maintained around 7.0 in the formation of the biofilm due to the non-cyclic continuous flow of the culture medium through the bacteria sample. The culture medium flow transported and diluted the acid substance produced by the bacteria out of the chamber 107 in formation of the biofilm, thereby maintaining the culture condition in the chamber 107 around 7.0. There is no need to add any alkaline substance to adjust the pH value of the culture condition, thereby reducing the possibility of contamination to the culture condition. It is similar to in vivo oral bacteria growth condition, thus it is able to generate the biofilm formed in vivo with the actual nature in vivo such as drug resistance, therefore it is able to precisely test the effect of the drugs on the biofilm formed in vivo.

The device 100 is efficient in screening drug candidate or testing the effect of a drug on inhibiting or preventing formation of biofilm. It takes only about 24 hours to complete one cycle of the test or screen. The use of the saliva collected from the human as the bacteria sample eliminates the step of the culturing the specific bacteria extracted from the salvia, thereby significantly reducing the test time.

In addition, the substrate 121 is coated with a coating layer (e.g., saliva protein layer), which promotes the inoculation of the source bacteria sample on the substrate 121 through absorbance force between the coating layer and the source bacteria sample, guaranteeing the formation of the biofilm in culture medium flow.

There is no need to add anaerobic gas into the device 100 described herein in culture when human saliva is taken as the source bacteria sample. Human saliva contains various oral bacteria including anaerobe and aerobe bacteria. In a culture process, the aerobe bacteria consume the oxides around the condition, thereby generating an anaerobic environment for the growth of the anaerobe bacteria. Therefore, there is no need to inlet anaerobic gas to create the anaerobic environment for the growth of the anaerobe, which is similar to the human oral cavity environment. Furthermore, the enclosed environment in the chamber and the flow of culture medium mimics the oral environment, the resulting biofilm (using saliva as the bacteria sample) mimic dental plaque accordingly.

In addition, the base 101 and the cover 151 are tightened and the chamber 107 is sealed, the components in the device can be autoclaved, the possibility of contamination to the biofilm (e.g., dental plaque) is significant reduced.

### Example 1

### Preparation and Validation Analysis of Dental Plaque Biofilm

A dental plaque biofilm was prepared as follows:

### Preparation of Culture Medium

The BMM was prepared according to the method described in Sissons, et al. J. Dent Res. 1991; 70(11): 1409-16. The components of the culture medium were as follows:

| | | |
|---|---|---|
| Hog gasric mucin | 2.5 | g/L |
| Proteose peptone | 10.0 | g/L |
| Trypicase peptone | 5.0 | g/L |
| Yeast extract | 5.0 | g/L |
| KCl | 2.5 | g/L |
| Haemin | 5 | mg/L |
| Arginine | 1 | mmol/L |
| L-cys | 0.1 | g/L |
| Urea | 1 | mmol/L |
| Vitamin K1 | 1 | mg/L |

Coating of the Substrate

60 ml saliva was collected from a volunteer and centrifuged. The supernatant fluid was collected and sterilized under ultraviolet irradiation. Total 16 hydroxyapatite slices were immersed in the sterilized supernatant fluid for at least 2 hours to make the hydroxyapatite slices be coated with the saliva protein layer.

Oral Bacteria Sample

4 ml saliva was collected from the volunteer and mixed with 5 ml BMM culture medium and 1 ml sucrose to obtain an oral bacteria sample.

Inoculation and Culture

4 devices each having therein 4 hydroxyapatite slices coated with the saliva protein layer were prepared. 2.5 ml saliva solution was added into each device. The culture medium was transported into the devices. The dilution rate of the culture medium was set to 0.6 h⁻¹.

Analysis of Dental Plaque Biofilm

Samples of the dental plaque biofilm were collected at 3 h, 6 h, 9 h, 12 h, 15 h, 18 h, 21 h and 24 h during the culture for the analysis.

1) Spectrophotometry Analysis: the hydroxyapatite slices were taken out from the devices and vibrated in 2 ml suspension liquid to obtain a suspension of the dental plaque biofilm samples. The optical density at 630 nm (the “OD₆₃₀”) of the samples was measured using a spectrophotometer. The samples were also spread on a spread plate to count the number of the bacteria colonies.

The results are shown in FIG. 7. According to the FIG. 7, the value of the OD₆₃₀ increased with time and had significant increase after 18 hours. Meanwhile, the Log CFU value of the dental plaque suspension became stable after 9 hours increase. This indicates that the amount of the live bacteria reached a stable state after 6 hours' culture while the extracellular metabolic product kept accumulating with time resulting in the increase of the dental plaque biofilm's volume.

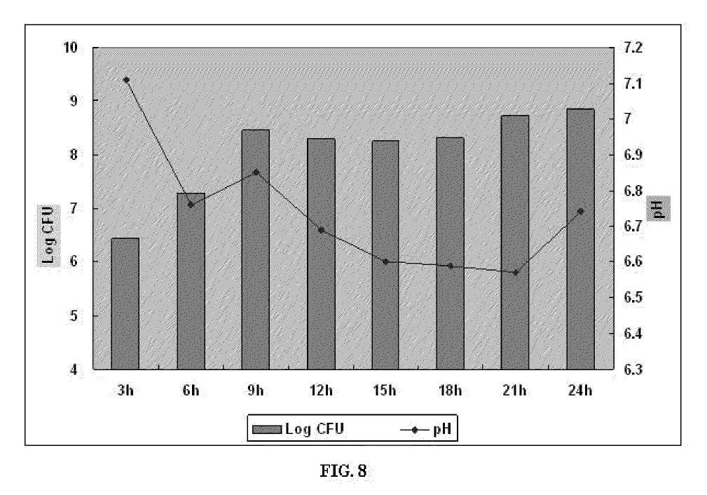

The samples of the bacteria liquid in the devices were also collected for Spectrophotometry analysis and pH value. The results are showed in FIG. 8. The concentration of the oral bacteria in the bacteria liquid becomes stable after 9 hours increase, which is similar to the dental plaque biofilm on the hydroxyapatite slices. It indicates that the bacteria in the bacteria liquid reached a balance with the bacteria of the dental plaque. In addition, the pH value kept in a stable range from 6.55 to 7.1 in the process of the culture. This range of the pH value is similar to the condition in the mouth and suitable for the growth of most of the oral bacteria. Moreover, there is no need to add any alkaline substance to adjust the pH value in the process of the culture, reducing the possibility of pollution in the test.

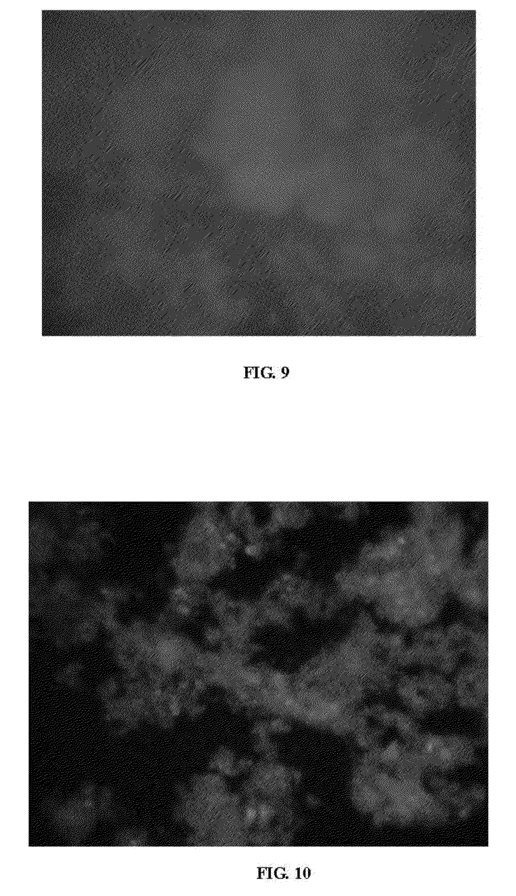

2) Fluorescent Microscope Analysis: a rapid fluorescence staining method using the LIVE/DEAD® Bacterial Viability Kit (BacLight™) was applied to distinguish the viability of bacteria in the dental plaque biofilm that grew on the HA slices for 24 hours. A dental plaque biofilm was stained without destroying the dental plaque biofilm. The fluorescence image of the biofilm (FIG. 9) shows that the dental plaque biofilm has a layer with compact structure. The surface of the dental plaque biofilm is covered with the dead bacteria. Another dental plaque biofilm was made into the homogenate before the test. The fluorescence image (FIG. 10) shows that most of the bacteria inside the dental plaque biofim are alive. The phenomenon that the dead bacteria was accumulated on the surface of the biofim and the live bacteria stayed inside of the dental plaque biofilm is consistent with the report in some references such asZaura-Arite E, van Mark J, ten Cate J M, Confocal microscopy study of undisturbed and chlorhexidine-treated dental biofilm. J Dent Res 80(5): 1436˜1440, 2001.

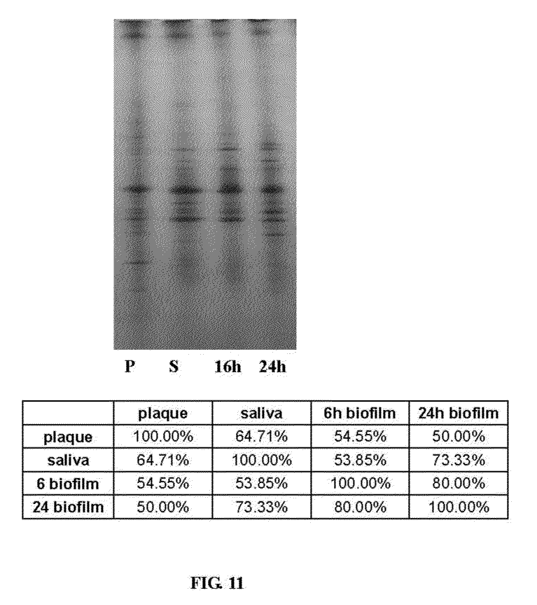

3) PCR-DGGE Analysis: The dental plaque biofilm samples were collected at 6 hours and 24 hours in the culture. The samples along with the saliva and the dental plaque were centrifuged at 12,000 rpm for 3 minutes, respectively. The precipitates were collected and washed with sterilized water for 3 times and then diluted to the concentration with OD₆₃₀=0.5. 1 ml of the solution were taken and centrifuged. The DNA is extracted according to the Shenggong kit G⁺ method. The PCR conditions were as follows. The initial denaturation was conducted under 94° C. for 3 min, and 35 cycles consisting of 1 min at 94° C., 1 min at 56° C., 2 min at 72° C., and an additional cycle of 5 min at 72° C. for chain elongation. The products were stained using the Bio-Rad silver stain kit. The result is shown in FIG. 11. The similarity of the four samples was calculated according to the formula as follows.

$$Similarity between A and B = \frac{Number of Same Band between A and B \times 2}{Number of Band A + Number of Band B} \times 100 \%$$

The results show that biofilm after 24 hours culture has high similarity to the original saliva, indicating that a biofilm with various species of bacteria can be obtained by culturing the original saliva in the device of the present disclosure.

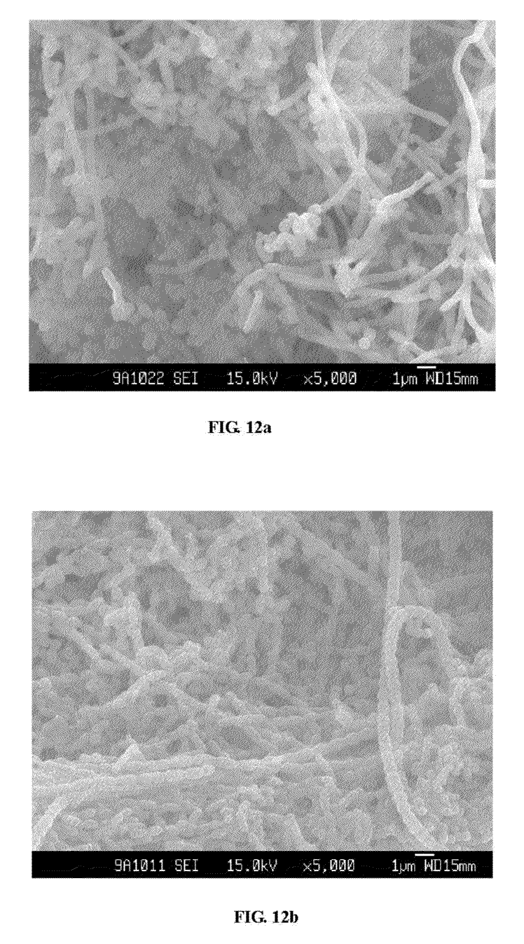

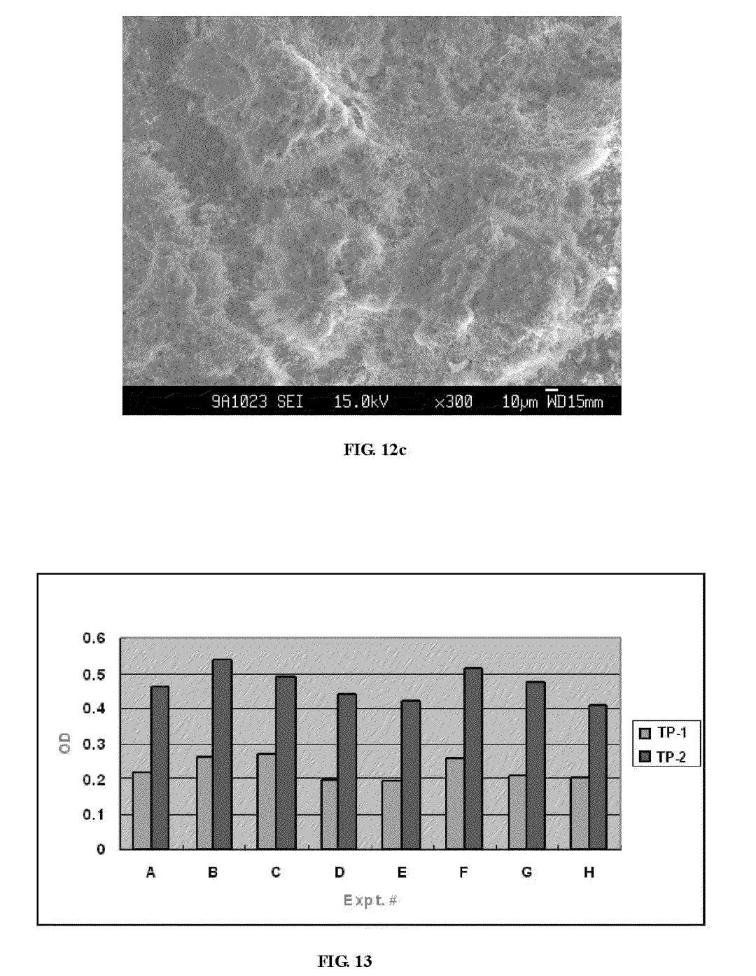

4) SEM Analysis: The dental plaque biofilm on the hydroxyapatite substrates after 24 hours' culture were subject to the SEM analysis. FIGS. 12a and 12b show images of the dental plaque with 5000 fold amplified. FIG. 12c shows an image of the dental plaque with 300 fold amplified. Various oral bacteria forms were observed in FIG. 12a and FIG. 12b, including the coccus, the bacillus, the clostridium and the filamentous bacteria. The majority of them are the coccus. This is consistent with the fact that the coccus has a high percentage of the bacteria in the oral cavity. In FIG. 12c, the microcolony that is particular in the formation process of the dental plaque biofilm is observed.

5) Selective Medium Culture Analysis: The samples of the dental plaque biofilm were collected after 6 h and 24 h culture for the selective medium culture. The samples of the saliva and the plaque collected from the volunteer were used for the comparison. The selective mediums used herein were columbia blood agar base (CA) for total amount of bacteria, CFAT agar (CFAT) for screening actinobacillus, MSA agar (MSA) for screening streptococcus, VA agar (VA) for screening veillonella, CVE agar for screening fusobacterium, lead acetate agar (PA) for identifying prevotella melaninogenica, negative bacteria blood agar for screening negative bacteria. The results are shown in the table 1. The results show that dental plaque biofilm cultured according to method of the present disclosure have various species of bacteria as the bacteria and the dental plaque collected from the human.

TABLE 1
Results of Selective Medium Culture Analysis

| Samples | | CA | PA | G-A | CVE | VA | CFAT | MSA |
|---|---|---|---|---|---|---|---|---|
| 0 h | Saliva (Log | 7.57 | 7.45 | 7.26 | 7.36 | 6.45 | 6.28 | 7.29 |
| | cfu/ml) | | | | | | | |
| | Plaque (Log | 8.29 | 8.08 | 7.76 | 7.90 | 7.41 | 6.69 | 7.62 |
| | cfu/ml) | | | | | | | |
| 6 h | HA (Log | 7.09 | 6.93 | 6.20 | 6.43 | 5.41 | 5.57 | 6.83 |
| 24 h | cfu/slice) | 8.45 | 8.43 | 7.91 | 8.07 | 7.40 | 6.34 | 8.06 |

The samples of the dental plaque biofilm prepared by the method of the present disclosure were also subject to the 16s rDNA gene sequence analysis for identification of bacteria on selective medium. The results shows that the dental biofilm includes the facultative anaerobes like Streptococcus. anginosus, Streptococcus. australis, Streptococcus. constellatus, Streptococcus. mitis, Enterobacter. sp, Actinomyces. sp and strict anaerobes like Veillonella. sp, Prevotella. melaminogenica, Fusobacterium. periodonticum.

### Example 2

### Test the Effect of Toothpastes on Dental Plaque

The effect of the COLGATE® total toothpaste with anti-plaque effect (TP-1) and an toothpaste without anti-plaque (TP-2) on the dental plaque were tested according to the method of the present disclosure. 1:2 volume ratio of the toothpaste to the water was mixed and used to treat the dental plaque after it was cultured for 8 hours. After 30 second treatment, the dental plaque was cultured again for 16 hours. After the culture, the dental plaque was subject to the spectrophotometry analysis. The test was repeated 8 times. The results were showed in the FIG. 13. According to the FIG. 13., the OD₆₃₀ values of the dental plaque biofilms that were treated by the TP-1 are all lower than the values of the biofilm that were treated by the TP-2. It indicates that the TP-1 has a good anti-plaque effect when it is compared to the TP-2. That is consistent with the results of the clinical test.

### Example 3

### Test the Effect of Mouthwash on Dental Plaque

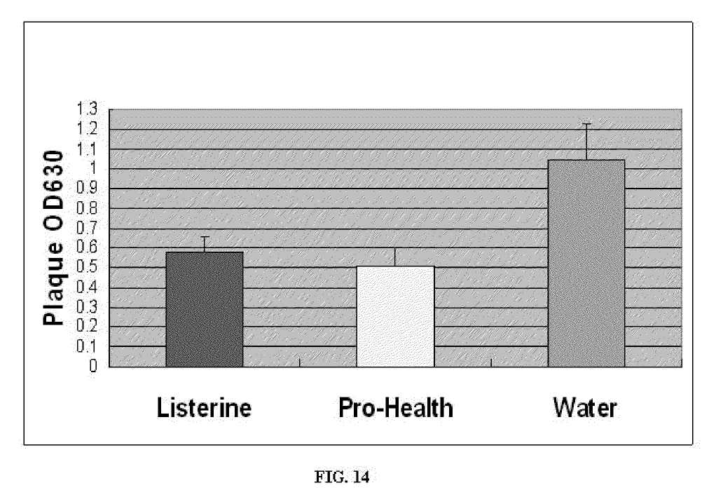

The effect of the Listerine mouthwash and Pro-Heath mouth on preventing the formation of the dental plaque is tested according to the method of the present disclosure. The water was used as a control. The mouthwash was used to treat the dental plaque directly for 1 minute after the dental plaque was cultured for 8 hours. After culture for another 16 hours, the dental plaque is subject to spectrophotometry analysis. The results were showed in the FIG. 14. According to the FIG. 14, the OD₆₃₀ values of the dental plaque biofilms that were treated by the mouth wash are both lower than the values of the biofilm that were treated by the water. It indicates that the mouthwash has a good anti-plaque effect when it is compared to the water, which is consistent with the fact.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to”, the term “having” should be interpreted as “having at least”, the term “includes” should be interpreted as “includes but is not limited to”, etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to disclosures containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

1. A device for forming a biofilm sample in vitro, comprising:
a base,
a cover,
a chamber defined by the base and the cover therebetween,
a concave structure formed on the bottom surface of the chamber, and
a first tube extending to the chamber and towards the concave structure, where the first tube connects the chamber and outside.

2. The device of claim 1, wherein the first tube is integrated on the cover.

3. The device of claim 1 further comprising a flexible pipe, wherein the flexible pipe connects to the first tube and extends to the bottom of the concave structure.

4. The device of claim 1, wherein the concave structure has an arc surface.

5. The device of claim 1 further comprising a position limiting structure and at least one substrate, wherein the position limiting structure is located in the chamber for securing said at least one substrate stable in the chamber.

6. The device of claim 1 further comprising a second tube connecting the chamber and outside, wherein the first tube and the second tube are located within the chamber but on opposite sides such that, when a liquid is introduced into the chamber through the first tube, the liquid passes across the chamber and elutes outside the chamber through the second tube.

7. The device of claim 1 further comprising a sealing member, wherein the sealing member surrounds the chamber and prevent a fluid from leaking out of the chamber when the base and the cover is tightened together.

8. The device of claim 8 wherein the sealing member is a rubber sealing ring.

9. The device of claim 1 wherein the base and cover are made of high temperature endurable and corrosion-resistant materials.

10. The device of claim 9 wherein the materials are selected from the group consisting of polymethylmethacrylate (PMMA), polycarbonate (PC), polypropylene (PP), polystyrene (PS), polyvinyl chloride (PVC), polyamide (PA) and a corrosion-resistant metal.

11. The device of claim 6 further comprising a second concave structure directed towards the second tube.

12. The device of claim 11 wherein the second tube is connected to a second flexible pipe and where the second flexible pipe extends to the bottom of the second concave structure substantially.

13. A system for forming biofilm samples, comprising:
a first according to claim 1 and a second device according to claim 1, and
a culture medium source in fluid communication with the first and the second devices simultaneously.

14. A system for forming biofilm samples, comprising:
a first device according to claim 1 and a second devices according to claim 1, and
a first culture medium source fluid communication with the first device and a second culture medium source in fluid communication with the second device.

15. The system of one of claims 13 and 14, wherein the first device contains a first source bacteria sample, and the second device contains a second source bacteria sample which is different from the first sample.

16. A system for testing the effect of drugs on biofilm, comprising:
a first and a second devices of claim 1, where the first and the second devices contain therein a first and a second biofilm samples, respectively, and
a first drug source in fluid communication with the first and the second devices simultaneous.

17. A system for testing the effect of drugs on biofilm, comprising:
a first and a second devices of claim 1, where the first and the second devices contain therein a first and a second biofilm samples, respectively, and
a first and a second drug sources in fluid communication with the first and the second devices, respectively.

18. A method for forming a biofilm sample, comprising:
inoculating a source bacteria sample on a substrate;
loading the substrate in the chamber of the device of claim 6; and
introducing a culture medium into the chamber through the first tube and eluding the culture medium outside the chamber through the second tube to allow the source bacteria sample to form a biofilm on the substrate.

19. The method of claim 18 further comprising a step of draining the culture medium in the chamber through the concave structure and the first tube.

20. The method of claim 18 further comprising: coating on the substrate with a coating layer prior to inoculation so that the source bacteria sample is inoculated on the coating layer.

21. The method of claim 18 wherein the source bacteria sample is selected from the group consisting of gram-positive bacteria, gram-negative bacteria, aerobic bacteria or anaerobic bacteria, or any combination thereof.

22. The method of claim 18 wherein the source bacteria sample is a saliva sample or a dental plaque sample or a tongue coating sample.

23. A biofilm composition comprising:
a substrate; and
a biofilm sample formed on the substrate, wherein the biofilm is formed using the method of claim 18.

24. The biofilm composition of claim 23 further comprising a coating layer formed between the substrate and the biofilm sample

25. The biofilm composition of claim 24 wherein the coating layer is selected from the group consisting of saliva protein, sterile saliva, mucin, adhesin, albumin, mucopolysaccharide, or any combination thereof.

26. The biofilm composition of claim 23 wherein the substrate is a piece of human tooth, a piece of animal tooth, a hydroxyapatite substrate, a fluorapatite substrate, resin substrate, polyolefin substrate, polystyrene substrate, polyvinyl chloride substrate or polyurethane substrate, metal discs substrate, marble substrate or glass discs substrate.

27. A method for testing a drug using the biofilm composition formed using the method of claim 18, comprising:
introducing a drug solution into the chamber through the first tube and eluding the drug solution outside the chamber through the second port to treat the biofilm sample with the drug solution; and
analyzing the drug treated biofilm sample.

28. An oral bacteria biofilm composition, comprising:
a substrate, and
an oral bacteria biofilm sample formed on the substrate,
wherein the biofilm sample is formed by directly dripping an oral bacterial sample on the substrate and then culturing the oral bacteria sample in a non-cyclic culture medium flow, where the oral bacteria sample is a saliva sample or a dental plaque sample or a tongue coating sample.

29. The model of claim 28, further comprising a coating solution layer between the substrate and the biofilm sample.

30. The model of claim 28, wherein the substrate is a piece of human tooth or a piece of animal tooth or a hydroxyapatite substrate or a fluorapatite substrate.

31. The model of claim 28, wherein the saliva or dental plaque or tongue coating sample is collected from a human being.

32. The model of claim 28, wherein the culture is conducted around 37° C.

33. A method of preparing an in vitro model of oral bacteria biofilm, comprising:
inoculating an oral bacteria sample to a substrate by directly dripping the oral bacteria sample on the substrate, and
culturing the oral bacteria sample in a non-cyclic culture medium flow to form an oral bacteria biofilm sample on the substrate,
where the oral bacteria sample is a saliva sample or a dental plaque sample or a tongue coating sample.

34. The method of claim 33 further comprising coating the substrate with a coating solution layer, where the oral bacteria sample is inoculated on the coating solution layer.

35. The method of claim 34, wherein the coating solution is saliva protein, sterile saliva, mucin, adhesin, albumin, or mucopolysaccharide.

36. The method of claim 33, wherein the substrate is a piece of human tooth or a piece of animal tooth or a hydroxyapatite substrate or a fluorapatite substrate.

37. The method of claim 33, wherein the saliva or dental plaque or tongue coating sample is collected from a human being.

38. A device for forming a biofilm sample in vitro, comprising:
a base,
a cover,
a chamber defined by the base and the cover therebetween, and
a first tube extending to a fluid collecting area in the chamber, which area a fluid in the chamber will flow to when the fluid needs to be drained out, where the first tube connects the chamber and outside.

39. The device of claim 38 further comprising a flexible pipe connecting to the first tube, where the flexible pipe extends to the fluid collecting area.

40. A method of preparing an in vitro model of biofilm, comprising:
inoculating a source bacteria sample to a substrate by directly dripping the source bacteria sample on the substrate, and
culturing the source bacteria sample in a non-cyclic culture medium flow to form a biofilm sample on the substrate.

41. The method of claim 40 further comprising coating the substrate with a coating solution layer, where the source bacteria sample is inoculated on the coating solution layer.
